(19) United States
(12) Patent Application Publication
(10) Pub. No.: US 2019/0000392 A1
(43) Pub. Date: Jan. 3, 2019

(54) Electronic Blood Pressure Monitor, Blood Pressure Measuring Method, and Electronic Stethoscope

(71) Applicant: Yamaha Corporation, Hamamatsu-shi (JP)

(72) Inventors: Yukitoshi SUZUKI, Hamamatsu-shi (JP); Yasuhiko SEKIMOTO, Hamamatsu-shi (JP); Morito MORISHIMA, Fukuroi-shi (JP)

(21) Appl. No.: 16/018,805

(22) Filed: Jun. 26, 2018

(30) Foreign Application Priority Data
Jun. 30, 2017 (JP) 2017-129489

(51) Int. Cl. A61B 5/00; A61B 5/022; A61B 7/02

(52) U.S. Cl. CPC A61B 5/7217; A61B 7/02; A61B 5/02208

(57) ABSTRACT

An electronic blood pressure monitor includes: a vibration sensor that includes a film shape, the vibration sensor detecting vibrations of a body surface, the vibration sensor converting the detected vibrations to an electrical signal corresponding to pressure generated in a thickness direction of the vibration sensor to output the electrical signal; and a stethoscope filter that passes a signal of a first predetermined frequency band among the output electrical signal, the first predetermined frequency band being determined based on a frequency characteristic of a stethoscope.

### CROSS-REFERENCE TO RELATED APPLICATION

Priority is claimed on Japanese Patent Application No. 2017-129489, filed Jun. 30, 2017, the content of which is incorporated herein by reference.

### BACKGROUND OF THE INVENTION

### Field of the Invention

The present invention relates to an electronic blood pressure monitor, a blood pressure measuring method, and an electronic stethoscope.

### Description of Related Art

Methods of measuring blood pressure include the Korotkoff method and the oscillometric method. In the Korotkoff method, the brachial artery is compressed by a cuff (arm band), and then a stethoscope is used to listen for vascular sounds (Korotkoff sounds) that are produced when the pressure of the cuff is released. The blood pressure value when the initial Korotkoff sounds are heard is the systolic blood pressure, and the blood pressure value when the Korotkoff sounds disappear is the diastolic blood pressure value. The oscillometric method is a method of measuring blood pressure using vibrations (pulse waves) occurring in the vessel wall when the cuff is depressurized instead of the Korotkoff sounds.

On the other hand, Japanese Examined Patent Application Publication No. H03-47087 (hereinafter Patent Document 1) discloses technology of detecting the aforementioned Korotkoff sounds and measuring blood pressure by electrical signal processing.

### SUMMARY OF THE INVENTION

However, since the oscillometric method measures blood pressure in a completely different way from the Korotkoff method, differences arise with the blood pressure measured by the Korotkoff method. In addition, in Patent Document 1, since blood pressure is measured merely by detecting sounds, blood pressure is measured on the basis of sounds that do not necessarily match the sounds obtained by a stethoscope.

The present invention has been made in view of such circumstances. An exemplary object of the present invention is to provide an electronic blood pressure monitor, a blood pressure measuring method, and an electronic stethoscope that measure blood pressure on the basis of the Korotkoff method, and can measure blood pressure based on sounds closer to the sounds obtained by a stethoscope.

An electronic blood pressure monitor according to an aspect of the present invention includes a vibration sensor that includes a film shape. The vibration sensor detects vibrations of a body surface. The vibration sensor converts the detected vibrations to an electrical signal corresponding to pressure generated in a thickness direction of the vibration sensor to output the electrical signal. The electronic blood pressure monitor further includes a stethoscope filter that passes a signal of a first predetermined frequency band among the output electrical signal. The first predetermined frequency band is determined based on a frequency characteristic of a stethoscope.

A blood pressure measuring method according to an aspect of the present invention includes: detecting, by a vibration sensor that comprises a film shape, vibrations of a body surface; converting, by the vibration sensor, the detected vibrations to an electrical signal corresponding to pressure generated in a thickness direction of the vibration sensor to output the electrical signal; passing, by a stethoscope filter, a signal of a predetermined frequency band among the output electrical signal, the predetermined frequency band being determined based on a frequency characteristic of a stethoscope.

An electronic stethoscope according to an aspect of the present invention includes a vibration sensor that includes a film shape. The vibration sensor detects vibrations of a body surface. The vibration sensor converts the detected vibrations to an electrical signal corresponding to pressure generated in a thickness direction of the vibration sensor to output the electrical signal. The electronic stethoscope further includes a stethoscope filter that passes a signal of a predetermined frequency band among the output electrical signal. The predetermined frequency band is determined based on a frequency characteristic of a stethoscope.

According to the present invention, blood pressure is measured based on the Korotkoff method, and it is possible to measure blood pressure on the basis of sounds closer to the sounds obtained by a stethoscope.

### BRIEF DESCRIPTION OF THE DRAWINGS

FIG. 1 is a configuration diagram showing a configuration example of an electronic blood pressure monitor 1 according to the first embodiment.

FIG. 2 is a diagram showing the characteristics of the vibration sensor 2 of the first embodiment.

FIG. 3A is a diagram showing an attachment example of the vibration sensor 2 of the first embodiment.

FIG. 3B is a diagram showing an attachment example of the vibration sensor 2 of the first embodiment.

FIG. 3C is a diagram showing an attachment example of the vibration sensor 2 of the first embodiment.

FIG. 3D is a diagram showing an attachment example of the vibration sensor 2 of the first embodiment.

FIG. 4 is a configuration diagram showing a configuration example of the stethoscope filter 4 of the first embodiment.

FIG. 5A is a diagram for describing the stethoscope 100.

FIG. 5B is a diagram for describing the stethoscope 100.

FIG. 6A is a diagram for describing the equivalent circuit of the chest piece filter unit 40 of the first embodiment.

FIG. 6B is a diagram for describing the equivalent circuit of the chest piece filter unit 40 of the first embodiment.

FIG. 7 is a diagram showing an example of the frequency characteristics of the filter of the chest piece filter unit 40 of the first embodiment.

FIG. 8 is a diagram for describing the tube filter unit 41 of the first embodiment.

FIG. 9 is a diagram that shows an example of the frequency characteristics of the filter of the tube filter unit 41 of the first embodiment.

FIG. 10 is a configuration diagram showing a configuration example of the loudness determiner 5 of the first embodiment.

FIG. 11 is a diagram showing an example of the loudness filter unit 50 of the first embodiment.

FIG. 12 is a flowchart showing the operation of the electronic blood pressure monitor 1 of the first embodiment.

FIG. 13 is a configuration diagram of a configuration example of the stethoscope filter 4A of the second embodiment.

FIG. 14 is a configuration diagram of a configuration example of the electronic stethoscope 10 of the third embodiment.

### DETAILED DESCRIPTION OF THE INVENTION

Hereinbelow, an electronic blood pressure monitor, a blood pressure measuring method, and an electronic stethoscope of the embodiments will be described referring to the diagrams.

### First Embodiment

First, the first embodiment will be described.

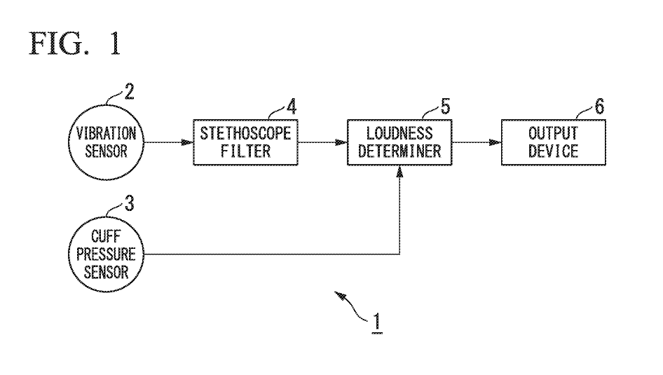

FIG. 1 is a configuration diagram showing a configuration example of an electronic blood pressure monitor 1 according to the first embodiment.

As shown in FIG. 1, the electronic blood pressure monitor 1 includes a vibration sensor 2, a cuff pressure sensor 3, a stethoscope filter 4, a loudness determiner 5, and an output device 6.

The vibration sensor 2 is a sensor that detects body surface vibrations, converts the detected vibrations to an electrical signal (hereinafter simply referred to as a signal), and outputs the electrical signal. For example, the vibration sensor 2 has a thin, soft, and light property in a mode in which an electret-converted porous organic material is formed into a film shape with electrodes formed on the front and back surfaces thereof. In the following, an example will be described in which the vibration sensor 2 is in the form of a film, but the present invention is not limited thereto. The vibration sensor 2 may be of any form provided detection of body surface vibrations is possible.

The vibration sensor 2 detects vibrations of the body surface by the occurrence of pressure in the thickness direction of the film surface of the vibration sensor 2 in accordance with vibrations of the body surface. The vibration sensor 2 outputs a signal corresponding to the detected body surface vibrations.

The cuff pressure sensor 3 detects the pressure (cuff pressure) when the upper arm is pressurized by a cuff attached to the upper arm of the person to be measured, converts the detected pressure to a signal, and outputs a signal. The cuff pressure sensor 3 detects, for example, the cuff pressure at predetermined time intervals. For example, the cuff pressure sensor 3 detects the cuff pressure during the process in which the upper arm is pressurized by the cuff and the cuff pressure during the process in which the upper arm is decompressed.

The stethoscope filter 4 passes a signal in a predetermined frequency band based on the frequency characteristics of a stethoscope from the signal output by the vibration sensor 2. “Frequency characteristics of a stethoscope” here means the relationship between the ratio of the intensity of the signal output from a stethoscope (output signal) to the intensity of the signal input to the stethoscope (input signal) and the frequency. The frequencies of the input signal and output signal of the stethoscope are frequencies of the audible band (for example, 20 Hz to 20 kHz) which can be perceived by human hearing.

The loudness determiner 5 determines the systolic blood pressure value and diastolic blood pressure value according to the Korotkoff method based on the signal output by the stethoscope filter 4 and the signal output by the cuff pressure sensor 3. For example, the loudness determiner 5, upon determining that Korotkoff sounds have started to be output by the stethoscope filter 4, determines the pressure indicated by the signal that is output by the cuff pressure sensor 3 when that determination is made to be the systolic blood pressure value. The loudness determiner 5, upon determining that the Korotkoff sounds output by the stethoscope filter 4 have ceased, determines the pressure indicated by the signal output by the cuff pressure sensor 3 when that determination is made to be the diastolic blood pressure value.

The output device 6 is, for example, a liquid crystal display that displays the systolic blood pressure value and the diastolic blood pressure value determined by the loudness determiner 5. The output device 6 may also be, for example, a speaker that reads out the systolic blood pressure value or the like. When the output device 6 is a speaker, the timing at which the systolic blood pressure value and the like are determined may be notified by an alarm sound. Moreover, the output device 6 may for example be a printer that prints the blood pressure values.

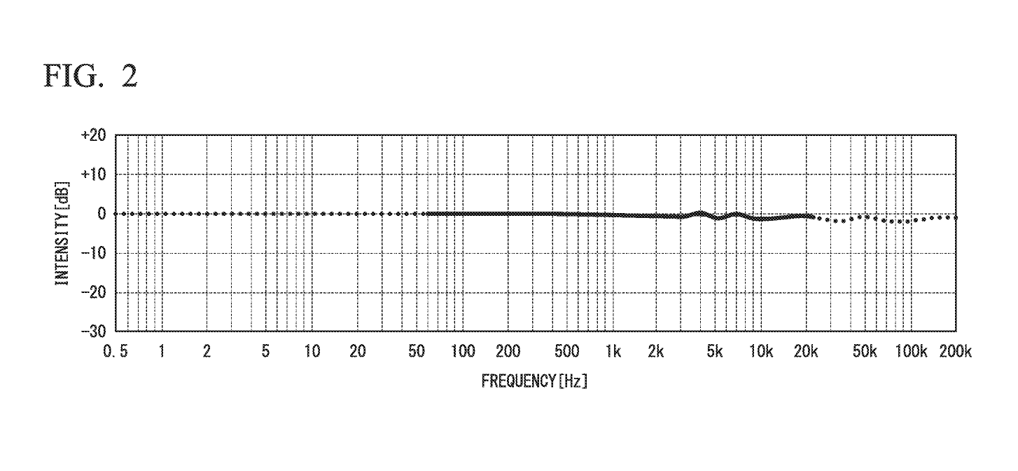

Next, the characteristics of the vibration sensor 2 will be described with reference to FIG. 2.

FIG. 2 is an example of the characteristics of the vibration sensor 2 of the first embodiment. FIG. 2 shows the relationship between the ratio of the intensity of the output signal of the vibration sensor 2 to the intensity of the vibration input to the vibration sensor 2, and frequency. In FIG. 2, the horizontal axis represents frequency (Hz) while the vertical axis represents signal intensity (dB).

Regarding the characteristics of the vibration sensor 2, as shown in FIG. 2, over a frequency range between about 0.5 Hz to about 200 kHz, a signal of nearly the same intensity as the intensity of the signal input to the vibration sensor 2 is output from the vibration sensor 2. In other words, in this frequency range, the vibration sensor 2 outputs a signal proportional to the magnitude of the vibration of the body surface.

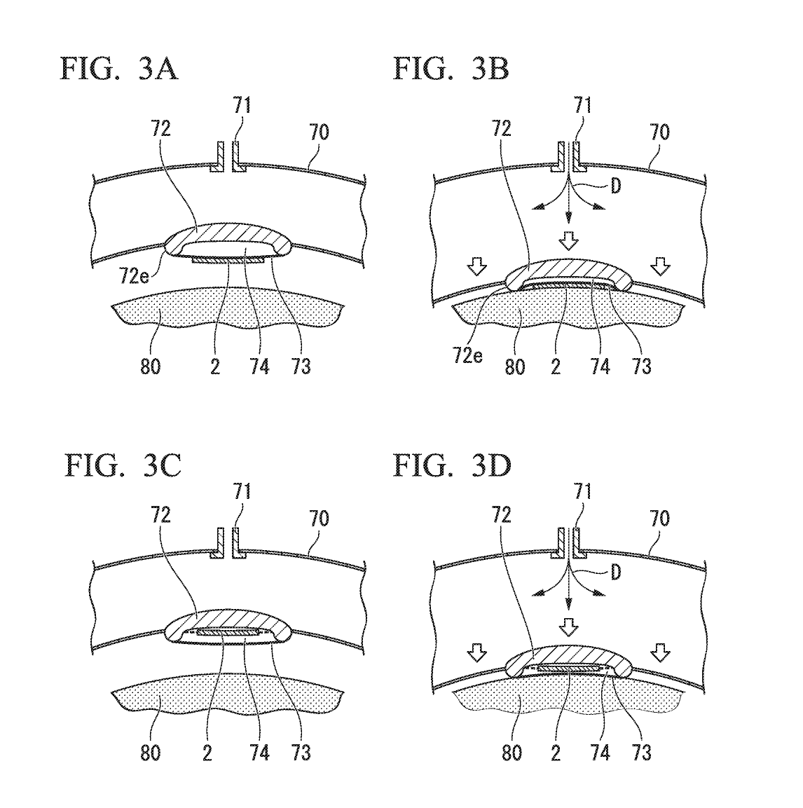

Next, an attachment example of the vibration sensor 2 will be described with reference to FIGS. 3A to 3D.

FIGS. 3A to 3D are diagrams showing an example of mounting of the vibration sensor 2 of the first embodiment. FIGS. 3A to 3D are sectional views in the circumferential direction of the upper arm in the state in which the cuff 70 is wrapped around the body surface 80.

FIG. 3A shows the state in which the body surface 80 is not pressurized by the cuff 70 in the example in which the vibration sensor 2 is attached so as to come in direct contact with the body surface 80. FIG. 3B shows a state in which the body surface 80 is pressurized by the cuff 70 in FIG. 3A.

FIG. 3C shows the state in which the body surface 80 is not pressurized by the cuff 70 in the example in which the vibration sensor 2 is attached via a diaphragm (membrane) 73 in contact with the body surface 80 so as to detect vibrations of the body surface 80. FIG. 3D shows the state in which the body surface 80 is pressurized by the cuff 70 in FIG. 3C.

As shown in FIG. 3A, the cuff 70 has a cuff pressure adjusting port 71 for adjusting the cuff pressure, a housing 72, the diaphragm 73, and an internal air chamber 74. The housing 72 has a concave shape on the side in contact with the body surface 80, with the diaphragm 73 stretched between the end portions 72e of the housing 72. The internal air chamber 74 is formed by the space surrounded by the concave portion inside the housing 72 and the diaphragm 73.

The vibration sensor 2 is connected to the surface of the diaphragm 73 which is in contact with the body surface 80.

As shown in FIG. 3B, air is introduced into the cuff 70 through the cuff pressure adjustment port 71 (reference symbol D), and when the body surface 80 is pressurized by the cuff 70, the end portions 72e are brought into contact with the body surface 80 and pressed thereagainst. In addition, as the end portions 72e are pressed against the body surface 80, the diaphragm 73 is pushed against the body surface 80 so as to sandwich the vibration sensor 2. As a result, the vibration sensor 2 is brought into close contact with the body surface 80 along the shape of the body surface 80. The vibration sensor 2 then detects vibrations of the body surface 80.

In the example of FIG. 3C, the vibration sensor 2 is accommodated between the housing 72 and the diaphragm 73. The vibration sensor 2 is attached at a position facing the diaphragm 73 on the inner peripheral surface of the concave portion of the housing 72.

As shown in FIG. 3D, when air is introduced into the cuff 70 through the cuff pressure adjustment port 71 (reference symbol D), the diaphragm 73 is pressed against the body surface 80. Thereby, the diaphragm 73 comes into close contact with the body surface 80 along the shape of the body surface 80. Then, the vibration sensor 2 detects the vibrations of the body surface 80 via the diaphragm 73 and the air in the internal air chamber 74.

In this manner, the vibration sensor 2 may be used in a state of directly contacting the body surface 80, or may be used in a state where it is not in direct contact with the body surface 80. The vibration sensor 2 detects vibrations of the body surface 80 when used in either of the states described above.

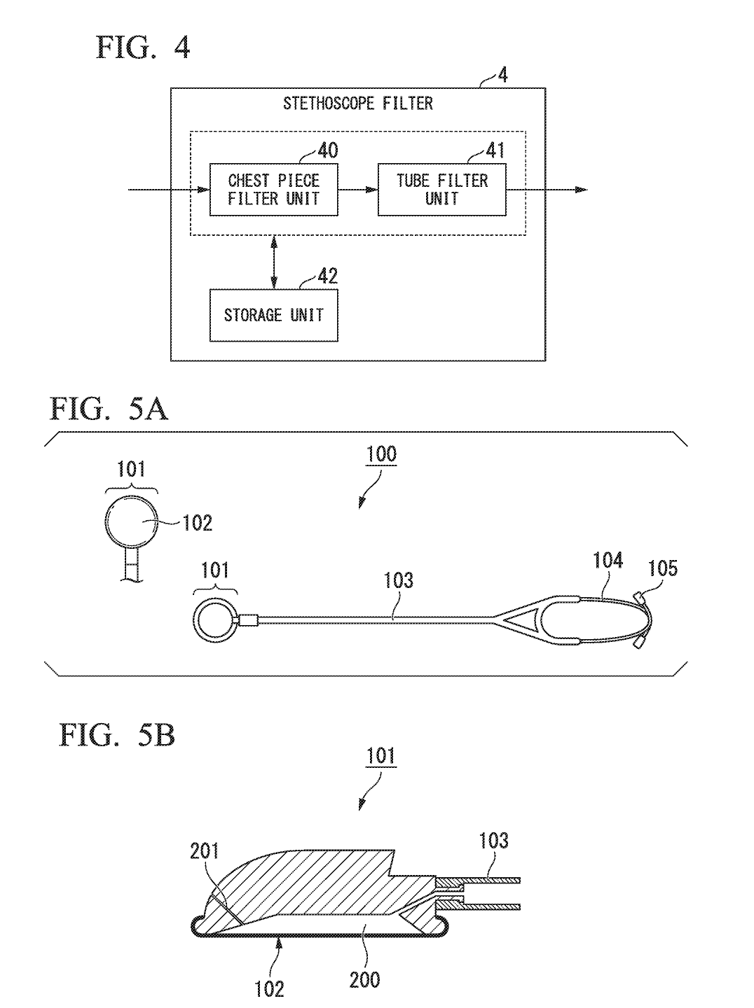

Next, a configuration example of the stethoscope filter 4 will be described with reference to FIG. 4.

FIG. 4 is a configuration diagram showing a configuration example of the stethoscope filter 4 of the first embodiment.

As shown in FIG. 4, the stethoscope filter 4 includes a chest piece filter unit 40, a tube filter unit 41, and a storage unit 42.

The stethoscope filter 4 is realized, for example, by a processor such as a CPU (central processing unit) executing a program stored in the storage unit 42. In addition, all or part of the stethoscope filter 4 may be realized by dedicated hardware such as large scale integration (LSI), an application specific integrated circuit (ASIC), a field-programmable gate array (FPGA), or the like.

The chest piece filter unit 40 is an example of the “first filter unit”, and the tube filter unit 41 is an example of the “second filter unit”.

Below, the chest piece filter unit 40 and the tube filter unit 41 will be described in turn.

First, the configuration of a stethoscope 100 will be described with reference to FIGS. 5A and 5B.

FIG. 5A is a diagram for explaining the stethoscope 100. FIG. 5A is a configuration diagram showing a configuration example of the stethoscope 100. FIG. 5B is a cross-sectional view of a chest piece 101 thereof in a plane perpendicular to the circumferential direction of a tube 103.

As shown in FIG. 5A, the stethoscope 100 includes, for example, the chest piece 101, the tube 103, an ear tube 104, and ear tips 105. The stethoscope 100 is a mechanical stethoscope without electrical signal processing.

A diaphragm 102 that is brought into contact with the body surface 80 is stretched across the chest piece 101. By placing the chest piece 101 on a living body to bring the diaphragm 102 into close contact with the body surface 80, vibrations of the body surface 80 are detected as vibrations of the diaphragm 102. The vibrations of the diaphragm 102 expand or compress the air in an internal air chamber 200 (see FIG. 5B). The sounds generated by expansion or compression of the air in the internal air chamber 200 are transmitted to the air that exists in the tube 103.

The tube 103 is a tube that connects the chest piece 101 and the ear tube 104, and transmits sounds based on the vibration detected by the diaphragm 102 to the ear tubes 104. The ear tube 104 connects between the tube 103 and the ear tips 105. The ear tube 104 has one end side (for example, for the right ear) and another end side (for example, for the left ear), with the ear tip 105 being attached to each. The sounds transmitted from the tube 103 are received by each ear tip 105. The ear tips 105 are inserted into the ears of the user of the electronic blood pressure monitor 1 and transmit the sounds transmitted from the ear tube 104 to the eardrums of the user.

As shown in FIG. 5B, the chest piece 101 has, for example, the diaphragm 102, the internal air chamber 200, and a vent hole 201.

The vent hole 201 brings the internal air chamber 200 into communication with the outside.

Next, an equivalent circuit of a chest piece filter unit 40 will be described with reference to FIGS. 6A and 6B.

FIG. 6A is a view for explaining the equivalent circuit of the chest piece filter unit 40 of the first embodiment.

FIG. 6A is an equivalent circuit based on the mechanical configuration of the chest piece 101. FIG. 6B is an equivalent circuit based on an electrical configuration corresponding to the equivalent circuit based on the mechanical configuration in FIG. 6B.

In FIG. 6A, it is assumed that the diaphragm 102 moves (curves) in the thickness direction at velocity Vm as a result of pressure Pm occurring in the direction of the arrow in FIG. 6A (the thickness direction of the diaphragm 102).

In the equivalent circuit shown in FIG. 6A, the driving of the diaphragm 102 in the direction of the arrow is affected by an elastic element (stiffness) 102Sm of the diaphragm 102, an inertial element 102Mm of the diaphragm 102, a mechanical resistive element 102Rm of the diaphragm 102, a mechanical elastic element (stiffness) 200Sm of the internal air chamber 200, and a mechanical resistive element 201Rm of the vent 201.

Here, the elastic element 102Sm is a variable indicating the relationship between the force acting on the diaphragm 102 and the elongation of the diaphragm 102 when the diaphragm 102 is for example expanded and contracted in the direction of the film surface. For example, the elastic element 102Sm is the elastic coefficient (spring constant) of the diaphragm 102. Assuming the diaphragm 102 to be a spring that expands and contracts in the direction of the film surface, the elastic element 102Sm in the equivalent circuit shown in FIG. 6A is a variable indicating a mechanical spring that expands and contracts with respect to pressure in a direction perpendicular to the thickness direction of the diaphragm 102 (the direction of the arrow in FIG. 6A).

The inertial element 102Mm is a variable indicating the relationship between the force acting on the diaphragm 102 and the displacement when the diaphragm 102 is for example driven in the thickness direction. The inertial element 102Mm is for example the mass of the diaphragm 102.

The resistive element 102Rm is a variable indicating the relationship between the force acting on the diaphragm 102 and the deformation amount when for example driving the diaphragm 102. The resistive element 102Rm is for example the viscous resistance of the diaphragm 102.

The elastic element 200Sm is a variable indicating the relationship between the force acting on the internal air chamber 200 and the expansion or compression amount of the internal air chamber 200 when expanding or compressing the air in the internal air chamber 200. The elastic element 200Sm is for example a spring constant of the air in the internal air chamber 200.

The resistive element 201Rm is a variable indicating the relationship between the force acting on the air existing in the vent hole 201 and the deformation amount of that air when causing air to pass to the outside through the vent hole 201. The resistive element 201Rm is for example the viscous resistance of the vent hole 201. The vent hole 201 has a function of causing the pressure of the internal air chamber 200 to follow the atmospheric pressure. For example, if the internal air chamber 200 were made a closed space, when the stethoscope 100 is used at a high altitude, a difference between the atmospheric pressure inside the internal air chamber 200 and the atmospheric pressure would occur, causing the diaphragm 102 to be pushed out by the air inside the chamber 200 and thereby be stretched. That is, the vent hole 201 serves to solve the problem of the tension of the diaphragm 102 being weakened due to elongation of the diaphragm 102 as a result of the pressure of the internal air chamber 200 being made to follow the atmospheric pressure when used at a high altitude or the like.

As shown in FIG. 6B, in the electrical equivalent circuit, the pressure Pm corresponds to the voltage Pe of the AC power supply that supplies power to the circuit. The velocity Vm corresponds to the current Ve flowing in the circuit.

Also, the elastic element 102Sm corresponds to the capacitor 102Se, the inertial element 102Mm corresponds to the coil 102Me, and the resistive element 102Rm corresponds to the resistor 102Re. The elastic element 200Sm corresponds to the capacitor 200Se, and the resistive element 201Rm corresponds to the resistor 201Re. The pressure in the internal air chamber 200 corresponds to the voltage P_{IR} indicating the potential difference between the positive electrode side and the negative electrode side of the capacitor 200Se.

When the voltage Pe is supplied to the equivalent circuit shown in FIG. 6B, the altered voltage P_{IR} results in accordance with the respective values of the capacitance of the capacitor 102Se, the inductance of the coil 102Me, the resistance of the resistor 102Re, the capacitance of the capacitor 200Se, and the resistance of the resistor 201Re.

As described above, in the present embodiment, the frequency characteristic of the filter of the chest piece filter unit 40 is expressed using an electrical equivalent circuit corresponding to the equivalent circuit based on the mechanical structure of the chest piece 101.

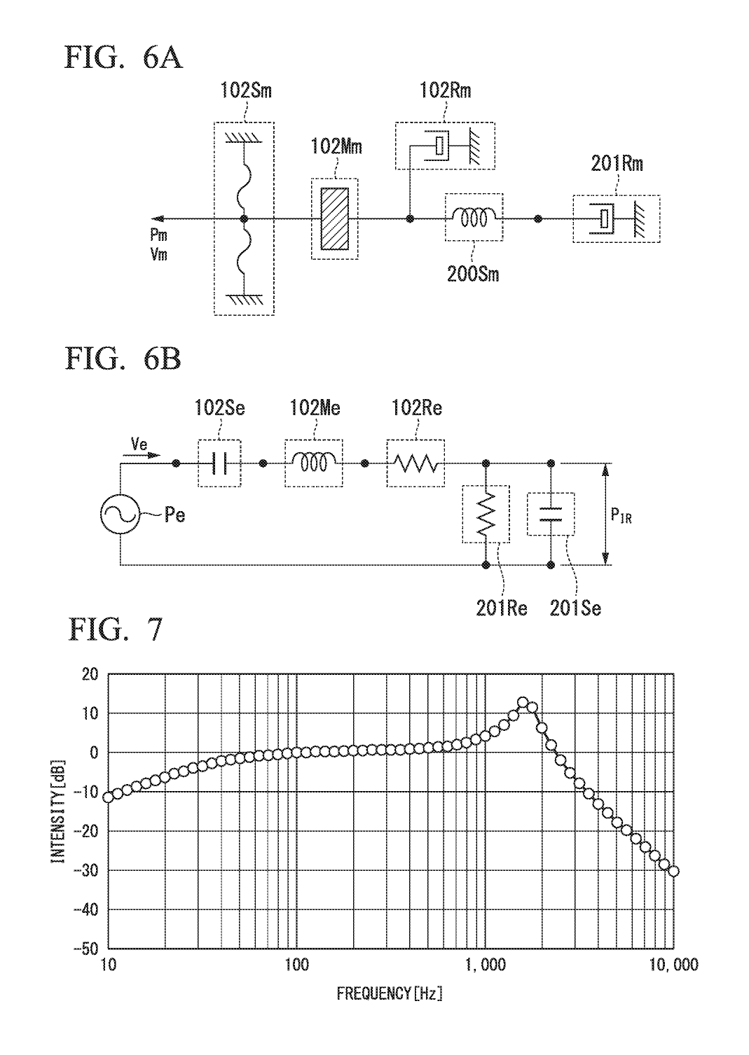

Next, the frequency characteristic of the filter of the chest piece filter unit 40 of the first embodiment will be described with reference to FIG. 7.

FIG. 7 is a diagram showing an example of the frequency characteristic of the filter of the chest piece filter unit 40 of the first embodiment. In FIG. 7, the horizontal axis represents frequency (Hz), and the vertical axis represents signal intensity (dB).

In the example of FIG. 7, the intensity is highest at a predetermined frequency (hereinafter referred to the peak frequency) in a range between 1,000 Hz and 2,000 Hz. This indicates that signals of the peak frequency are most likely to pass in the frequency characteristic of the filter of the chest piece filter unit 40. Moreover, in the example shown in FIG. 7, at frequencies higher than the peak frequency signals do not pass easily.

In the example of FIG. 7, although signals at a frequency lower than the peak frequency, specifically, in the frequency range of 20 Hz to 1,000 Hz, have a lower intensity than signals of the peak frequency, the signals still pass with a nearly uniform intensity.

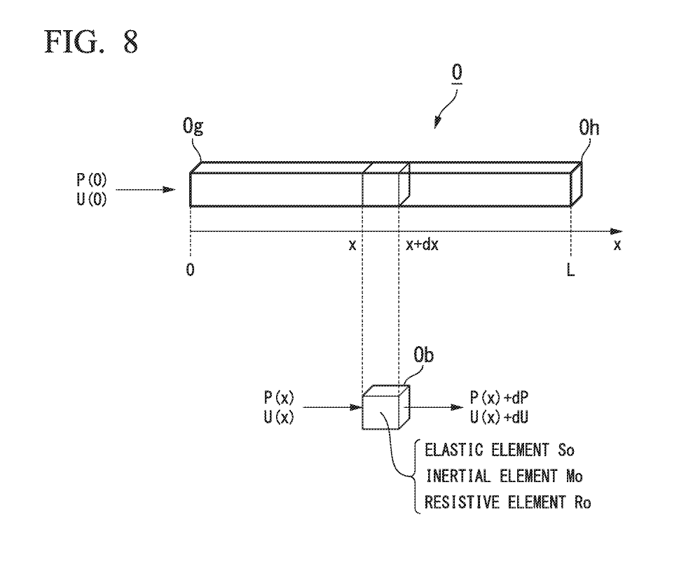

Next, the tube filter unit 41 will be described with reference to FIG. 8.

FIG. 8 is a diagram for explaining the tube filter unit 41 of the first embodiment. FIG. 8 is a model of the tube filter unit 41 based on the acoustic structure of the tube unit of the stethoscope 100. “Tube unit” of the stethoscope 100 is a generic term collectively referring to the tube 103, the ear canal 104, and the ear tips 105. The action of mechanical pressure in the model based on the mechanical structure shown in FIG. 6B corresponds to the action of sound pressure in the model based on the acoustic structure shown in FIG. 8. Further, the velocity at which mass is driven in the model based on the mechanical structure shown in FIG. 6B corresponds to the volume velocity when the medium that carries sound (for example, air) in the model based on the acoustic structure shown in FIG. 8 is driven.

In the model shown in FIG. 8, an acoustic tube O with length L is assumed. Here, “length L” in the model shown in FIG. 8 corresponds to the length of the above-mentioned “tube unit” of the stethoscope 100. An opening end Og on one side of the acoustic tube O corresponds to the end portion at which the tube 103 is connected to the chest piece 101. The closed end Oh on the other side of the acoustic tube O corresponds to the position of the end portion on the eardrum side of the ear tip 105.

As shown in FIG. 8, the longitudinal axis direction of the acoustic tube O is taken as the direction of the coordinate system x-axis. The x coordinate value of the opening end Og is 0 (zero), while the x coordinate of the closed end Oh is L.

In the model shown in FIG. 8, the sound pressure P(x) acting on the center of a unit of air (hereinafter simply referred to as an air unit) Ob having a certain volume is received and driven at the volume velocity U(x) in the x-axis direction. When the air unit Ob is driven in the x-axis direction, it is influenced by the elastic element So, the inertial element Mo, and the resistive element Ro of the air unit Ob.

The elastic element So of the air unit Ob is a variable indicating the relationship between the force applied to the air unit Ob and the expansion or compression amount when the air unit Ob is expanded or compressed. For example, the elastic element So of the air piece Ob is the elastic coefficient (spring constant) of the air unit Ob.

The inertial element Mo of the air unit Ob is a variable indicating the relationship between the force acting on the air unit Ob and the displacement amount when the air unit Ob is driven. For example, the inertial element Mo of the air unit Ob is the mass of the air unit Ob.

The resistive element Ro of the air unit Ob is a variable indicating the relationship between the force acting on the air unit Ob and the deformation amount when the air unit Ob is driven. For example, the resistive element Ro of the air unit Ob is the viscous resistance of the air unit Ob.

In the electrical equivalent circuit, the sound pressure P(x) and the volume velocity U(x) in the model described above correspond to voltage and current, respectively. In the model described above, the elastic element So corresponds to a capacitor, the inertial element Mo corresponds to a coil, and the resistive element Ro corresponds to a resistor.

The propagation coefficient of the electrical equivalent circuit corresponding to the model shown in FIG. 8 is, for example, expressed by the following Equation (1). Here, M is the mass per unit length of air in the acoustic tube O (acoustic mass), C_{A} is the capacity per unit length of air in the acoustic tube O (air capacity), R_{A} is the resistance per unit length of air in the acoustic tube O (acoustic resistance), and G_{A} is the conductance per unit length of air in the acoustic tube O (acoustic conductance).

γ_{A}=\sqrt{(R_{A}+jωM)(G_{A}+jωC_{A})} EQUATION (1)

The impedance (acoustic impedance) of the electrical equivalent circuit corresponding to the model shown in FIG. 8 is represented by for example the following Equation (2). In the equation, M is the acoustic mass per unit length of air in the acoustic tube O, C_{A} is the acoustic capacity per unit length of air in the acoustic tube O, R_{A} is the acoustic resistance per unit length of air in the acoustic tube O, and G_{A} is the acoustic conductance per unit length of air in the acoustic tube O.

$${Z}_{0 A} = \sqrt{\frac{{R}_{A} + j ω M}{{G}_{A} + j ω {C}_{A}}} \qquad EQUATION ( 2 )$$

In this way, in the present embodiment, the frequency characteristics of the filter of the tube filter unit 41 are, for example, represented by an electrical equivalent circuit corresponding to the model based on the acoustic structure of the tube of the stethoscope 100 as shown in FIG. 8.

In the equivalent circuit of the tube filter unit 41, the inertial element, the elastic element, and the resistive element of the surface along the inner diameter of the tube unit may be further added.

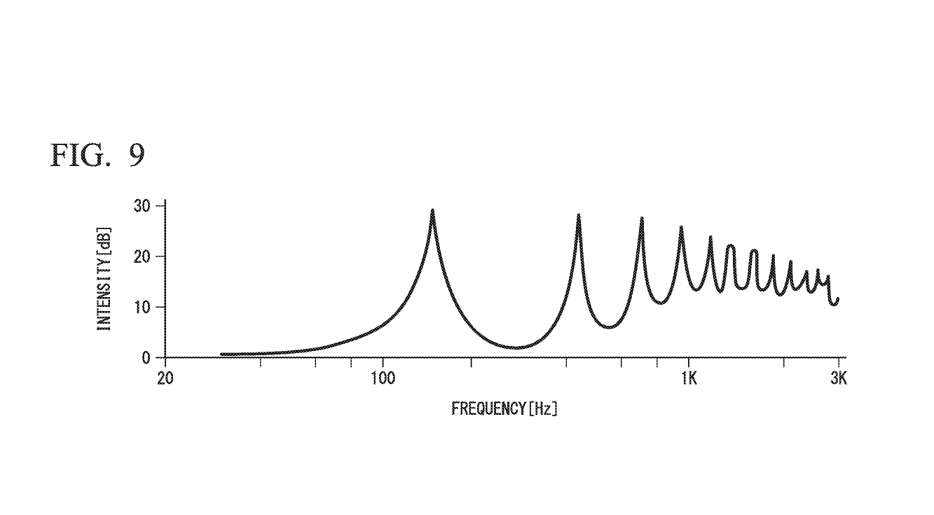

FIG. 9 is a diagram showing an example of the frequency characteristics of the filter of the tube filter unit 41 of the first embodiment. In FIG. 9, the horizontal axis represents the frequency (Hz), and the vertical axis represents the intensity (dB) of the output signal. FIG. 9 shows an example of the case in which the volume velocity U is 0 at the position coordinate x=L in the model shown in FIG. 8.

In the example of FIG. 9, the output from the tube filter unit 41 has such a characteristic that the band in which the sound pressure increases and the band in which the sound pressure decreases are periodically repeated according to the frequency. In the example of FIG. 9, when the length L of the acoustic tube O is an integral multiple of ¼ of the wavelength λ of the output signal, the sound pressure increases at the position coordinate x=L. In this case, the length of the tube filter unit 41 is set so that the intensity (volume) of the signal at which the integral multiple of ¼ of the wavelength λ is L at the position of the eardrum increases.

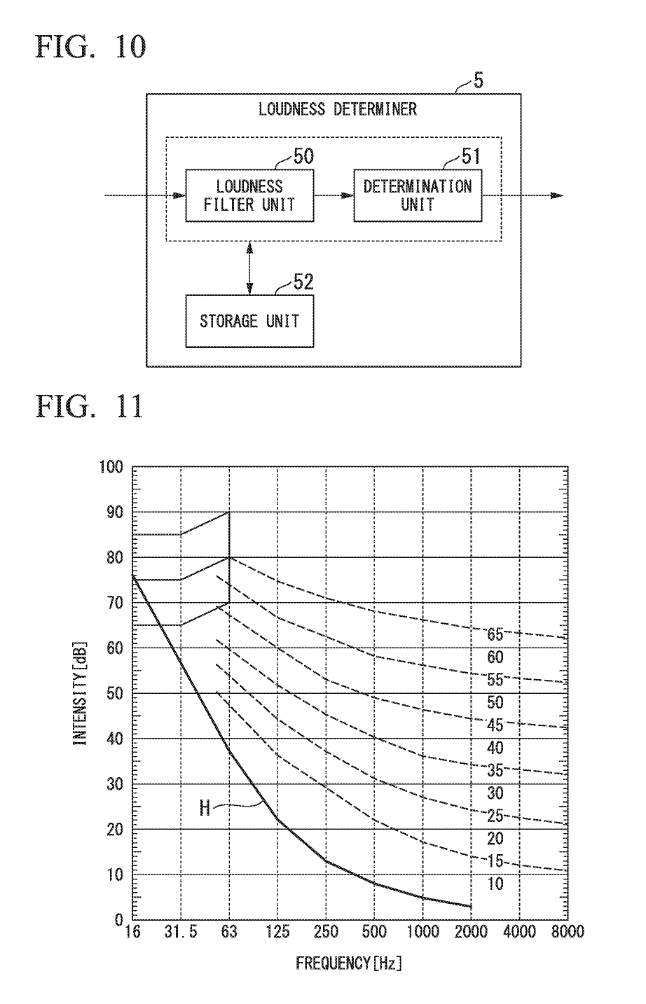

Next, a configuration example of the loudness determiner 5 will be described with reference to FIG. 10.

FIG. 10 is a configuration diagram showing a configuration example of the loudness determiner 5 of the first embodiment.

As shown in FIG. 10, the loudness determiner 5 includes a loudness filter unit 50, a determination unit 51, and a storage unit 52.

The loudness determiner 5 is implemented by, for example, a processor such as a CPU executing a program stored in the storage unit 52. In addition, all or part of the loudness determiner 5 may be realized by dedicated hardware such as LSI, ASIC, or FPGA.

The loudness filter unit 50 passes a signal of the predetermined frequency band based on the auditory characteristics of a person from the signal output by the stethoscope filter 4. The auditory characteristics of a person indicate for example the relationship between the minimum sound pressure that can be heard by a person having normal or general hearing ability and frequency.

The determination unit 51 determines whether Korotkoff sounds are included in the signal from the loudness filter unit 50 based on the amplitude of the signal from the loudness filter unit 50. For example, the determination unit 51 compares a predetermined threshold value with the amplitude of the signal from the loudness filter unit 50. When the amplitude of the signal from the loudness filter unit 50 is equal to or greater than the threshold value, the determination unit 51 determines that Korotkoff sounds are included. When the amplitude of the signal from the loudness filter unit 50 is less than the threshold value, the determination unit 51 determines that Korotkoff sounds are not included.

Further, the determination unit 51 determines the systolic blood pressure value and the diastolic blood pressure value based on the determination result concerning whether Korotkoff sounds are included, and the pressure value indicated by a signal output by the cuff pressure sensor 3. The determination unit 51 determines the systolic blood pressure value as the blood pressure value corresponding to the output value from the cuff pressure sensor 3 at the time of the transition from the state in which Korotkoff sounds are determined not to be included to the state in which Korotkoff sounds are determined to be included. Further, the determination unit 51 determines the diastolic blood pressure value as the blood pressure value corresponding to the output value from the cuff pressure sensor 3 at the time of the transition from the state in which Korotkoff sounds are determined to be included to the state in which Korotkoff sounds are determined not to be included.

Next, the frequency characteristics of the filter of the loudness filter unit 50 will be described with reference to FIG. 11.

FIG. 11 is a diagram showing an example of characteristics of the loudness filter unit 50. In FIG. 11, the horizontal axis represents the center frequency (Hz), and the vertical axis represents the intensity (dB) of the sound pressure of the output signal. In the example of FIG. 11, the frequency characteristics of the filter of the loudness filter unit 50 are determined on the basis of a waveform showing the relationship between the minimum sound pressure that can be heard by a person having normal or general hearing ability and frequency, and here corresponds to the loudness curve H.

The loudness filter unit 50 passes a signal having a higher signal strength than the curve shown in FIG. 11 and blocks a signal having a lower signal strength than the curve shown in FIG. 11. For example, when a signal having a frequency of 500 Hz is input to the loudness filter unit 50 with a signal strength of 10 dB, the loudness filter unit 50 passes this input signal. In addition, when a signal having a frequency of 250 Hz is input to the loudness filter unit 50 with a signal strength of 10 dB, the loudness filter unit 50 blocks the input signal.

As described above, the electronic blood pressure monitor 1 of the first embodiment includes the vibration sensor 2 that detects the vibration of the body surface, converts the detected vibration into an electrical signal, and outputs the electrical signal, and the stethoscope filter 4 that passes a signal of a predetermined frequency band determined based on the frequency characteristics of the stethoscope 100 from the electrical signal output by the vibration sensor 2.

As a result, in the electronic blood pressure monitor 1 of the first embodiment, blood pressure measurement is possible based on the Korotkoff method, and it is possible to measure blood pressure based on sounds closer to the sounds obtained by a stethoscope (a sound equivalent to that obtained by the stethoscope). That is, it is possible to measure the sounds of the brachial artery with the vibration sensor 2, and it is possible to remove unnecessary sounds among the detected vascular sounds and output Korotkoff sounds that are closer to the sounds obtained by a stethoscope. Then, by measuring the blood pressure based on the Korotkoff sounds output from the stethoscope filter 4, it is possible to measure the blood pressure based on the Korotkoff method.

In an electronic blood pressure monitor using the Korotkoff method, if a microphone is used instead of a stethoscope, the microphone picks up external noise in addition to vascular sounds, resulting in the detection of sounds differing from those when a stethoscope is placed on the upper arm.

On the other hand, in the electronic blood pressure monitor 1 of the first embodiment, since the vibration sensor 2 is used, it is possible to detect vascular sounds by detecting the vibration of the upper arm, and it is possible to detect sounds closer to the sounds obtained by a stethoscope.

In the electronic blood pressure monitor 1 of the first embodiment, the vibration sensor 2 is a film-like sensor that outputs an electrical signal corresponding to the pressure generated in the thickness direction of the film. Thereby, in the electronic blood pressure monitor 1 of the first embodiment, the sounds detected by bringing the diaphragm 102 of the stethoscope 100 into contact with the body surface can be detected by bringing the film surface of the vibration sensor 2 into contact with the body surface. As a result, it is possible to measure blood pressure on the basis of sounds closer to the sounds obtained by a stethoscope.

In the electronic blood pressure monitor 1 of the first embodiment, the stethoscope filter 4 has a chest piece filter unit 40 that passes a signal of a predetermined frequency band determined based on the characteristics of the chest piece 101. Thereby, the electronic blood pressure monitor 1 of the first embodiment can correspond to the characteristics of the chest piece 101, and the frequency characteristics of the stethoscope 100 can be more accurately reproduced. As a result, it is possible to measure blood pressure on the basis of sounds closer to the sounds obtained by a stethoscope.

Further, in the electronic blood pressure monitor 1 of the first embodiment, the chest piece filter unit 40 passes a signal of a predetermined frequency band on the basis of at least one of the inertial element of the diaphragm 102, the elastic element of the diaphragm 102, the resistive element of the diaphragm 102, the elastic element of the internal air chamber 200, and the resistive element of the internal air chamber 200. Thereby, with the electronic blood pressure monitor 1 of the first embodiment, the frequency characteristics of the stethoscope can be more accurately reproduced in accordance with the respective characteristics of the diaphragm 102 and the internal air chamber 200 constituting the chest piece 101. As a result, it is possible to measure blood pressure on the basis of sounds closer to the sounds obtained by a stethoscope.

Further, in the electronic blood pressure monitor 1 of the first embodiment, the chest piece filter unit 40 passes a signal of a predetermined frequency band on the basis of the resistive element of the vent hole 201. Thereby, in the electronic blood pressure monitor 1 of the first embodiment, when the vent hole 201 is present in the chest piece 101, the frequency characteristics of the stethoscope 100 can be more accurately reproduced according to the characteristics of the vent hole 201, and so it is possible to measure blood pressure on the basis of sounds closer to the sounds obtained by a stethoscope.

In the electronic blood pressure monitor 1 according to the first embodiment, the stethoscope filter 4 has a tube filter unit 41 that passes a signal of a predetermined frequency band determined based on the characteristics of the tube unit of the stethoscope 100. Thereby, the electronic blood pressure monitor 1 of the first embodiment can be made to correspond to the characteristics of the tube unit of the stethoscope 100, and so the frequency characteristics of the stethoscope 100 can be more accurately reproduced. As a result, it is possible to measure blood pressure on the basis of sounds closer to the sounds obtained by a stethoscope.

In the electronic blood pressure monitor 1 of the first embodiment, the tube filter unit 41 passes a signal of a predetermined frequency band on the basis of at least one of the elastic element of the medium transmitting sound in the space formed by one end portion and the other end portion of the surface along the inner diameter of the tube unit of the stethoscope 100, and an inertial element of the medium. Thereby, in the electronic blood pressure monitor 1 of the first embodiment, the frequency characteristics of the stethoscope 100 can be more accurately reproduced in accordance with the characteristics of the tube unit of the stethoscope 100. As a result, it is possible to measure blood pressure on the basis of sounds closer to the sounds obtained by the stethoscope.

The electronic blood pressure monitor 1 according to the first embodiment further includes a loudness filter unit 50 that passes a signal of a predetermined frequency band determined based on human auditory characteristics from the electrical signal output by the stethoscope filter 4. Thereby, the electronic blood pressure monitor 1 of the first embodiment can more accurately reproduce the sounds output from the stethoscope 100 that a person's ears can perceive, and so can measure blood pressure on the basis of sounds closer to the sounds obtained by a stethoscope.

Next, the operation of the electronic blood pressure monitor 1 of the first embodiment will be described.

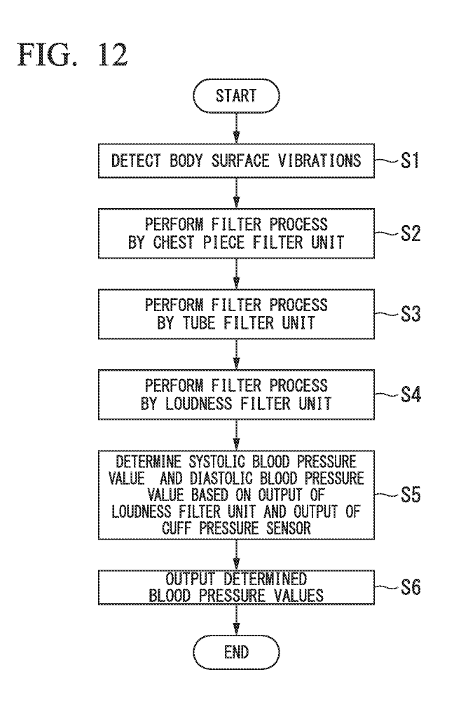

FIG. 12 is a flowchart showing the operation flow of the electronic blood pressure monitor 1 according to the first embodiment.

First, in the electronic blood pressure 1, the vibration sensor 2 detects vibrations of the body surface of the subject (Step S1). The vibrations of the body surface detected by the vibration sensor 2 are converted to an electrical signal and input to the stethoscope filter 4.

Next, in the electronic blood pressure monitor 1, the chest piece filter unit 40 performs filter processing (Step S2). The signal output from the chest piece filter unit 40 is a signal obtained by passing a signal of a predetermined frequency band determined based on the characteristics of the chest piece 101 from the electrical signal based on the vibrations of the body surface.

Next, in the electronic blood pressure monitor 1, the tube filter unit 41 performs filtering processing (Step S3). The signal output from the tube filter unit 41 is a signal obtained by passing a signal of a predetermined frequency band determined based on the characteristics of the chest piece 101 from the signal output from the chest piece filter unit 40.

Next, in the electronic blood pressure monitor 1, the loudness filter unit 50 performs filter processing (Step S4). The signal output from the loudness filter unit 50 is a signal obtained by passing the signal of a sound louder than the minimum sound that can be perceived by human hearing.

Moreover, in the electronic blood pressure monitor 1, the determination unit 51 determines the systolic blood pressure value and the diastolic blood pressure value based on the signal output from the loudness filter unit 50, which indicates sounds that can be heard by a person with a stethoscope, and a signal indicating the cuff pressure output from the cuff pressure sensor 3 (Step S5).

In the electronic blood pressure monitor 1, the output device 6 outputs the blood pressure values determined by the determination unit 51 (Step S6).

### Second Embodiment

Next, the second embodiment will now be described.

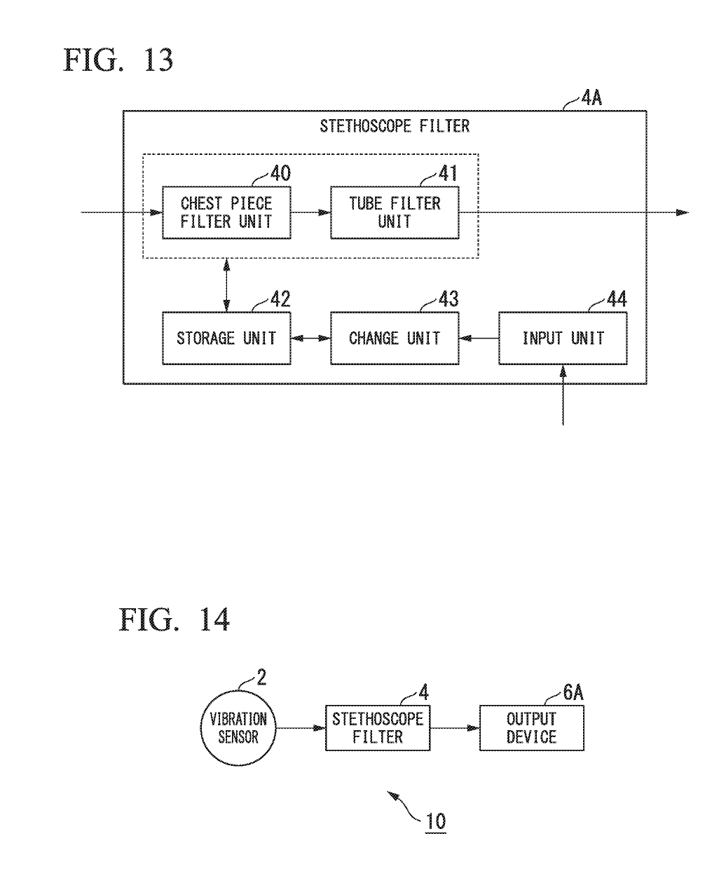

FIG. 13 is a configuration diagram showing a configuration example of a stethoscope filter 4A of the second embodiment.

In the second embodiment, the stethoscope filter 4A differs from the stethoscope filter 4 of the first embodiment by being provided with a change unit 43 and an input unit 44. Constitutions other than those described below are the same as those in the first embodiment described above.

In the second embodiment, the stethoscope filter 4A changes the frequency characteristics of the respective filters of the chest piece filter unit 40 and the tube filter unit 41 in accordance with an external instruction. For example, when blood pressure is measured using a specific stethoscope, the stethoscope filter 4A changes the frequency characteristics of the respective filters of the chest piece filter unit 40 and the tube filter unit 41 in accordance with the characteristics of that specific stethoscope. This makes it possible to measure blood pressure in a state close to the case where blood pressure is measured using the specific stethoscope.

As shown in FIG. 13, the stethoscope filter 4A is provided with the chest piece filter unit 40, the tube filter unit 41, the change unit 43, and the input unit 44.

The input unit 44 acquires change information input from an external device. The external device here is, for example, an information processing device such as a personal computer. The external device may input the change information to the input unit 44 via a network. The change information here is, for example, a circuit constant of the chest piece filter unit 40 or the tube filter unit 41. The circuit constant includes at least one of the coil's inductance value, the capacitor's capacitance value, and the resistor's resistance value in the equivalent circuit of each filter.

On the basis of a change signal from the input unit 44, the change unit 43 rewrites an execution program, stored in the storage unit 42, for causing the chest piece filter unit 40 or the tube filter unit 41 to execute the filter process. The change unit 43 may also rewrite variables such as circuit constants stored in the storage unit 42.

In the electronic blood pressure monitor 1 of the second embodiment as described above, the stethoscope filter 4 is further provided with input units 44 and 54 for inputting change information indicating a change of the respective circuit constants of the chest piece filter unit 40 and the tube filter unit 41 (which are examples of a “filter passing a signal in a predetermined frequency band”), and change units 43 and 53 for changing the circuit constants on the basis of the change information input by the input units 44 and 54. Thereby, in the electronic blood pressure monitor 1 of the second embodiment, it is possible to reproduce the sounds audible from each stethoscope in accordance with the characteristics of each stethoscope, and it is possible to measure blood pressure on the basis of sounds closer to the sounds obtained by the respective stethoscopes.

### Third Embodiment

The third embodiment will now be described.

FIG. 14 is a configuration diagram showing a configuration example of an electronic stethoscope 10 according to the third embodiment.

The third embodiment differs from the above-mentioned embodiments in that the electronic stethoscope 10 does not include the cuff pressure sensor 3 and the loudness determiner 5. Further, in the third embodiment, the electronic stethoscope 10 differs from the above-described embodiments in that the electronic stethoscope 10 includes an output device 6A in place of the output device 6 described above. Constitutions other than those described below are the same as those in the above-described embodiments. That is, in the present embodiment, some constitutions of the blood pressure monitor can be used as the electronic stethoscope.

The output device 6A outputs the electrical signal output by the stethoscope filter 4. The output device 6A is, for example, a liquid crystal display for displaying the waveform of the electrical signal output by the stethoscope filter 4. The output device 6A may for example be a speaker that outputs sound based on the electrical signal output by the stethoscope filter 4. The output device 6A may also for example be a printer that prints the waveform of the electrical signal output by the stethoscope filter 4. By using the cuff 70 and the electronic stethoscope 10, the user of the electronic stethoscope 10, while referring to the output of the electronic stethoscope 10 when measuring the blood pressure of a person to be measured, can determine the systolic blood pressure value and diastolic blood pressure value by referring to the cuff pressure.

As described above, the electronic stethoscope 10 according to the third embodiment includes the vibration sensor 2 that detects vibrations of the body surface, converts the detected vibrations into an electrical signal, and outputs the electrical signal, and the stethoscope filter 4 that passes a signal of a predetermined frequency band based on the frequency characteristics of the stethoscope from the electrical signal output by the vibration sensor 2.

As a result, in the electronic stethoscope 10, the vibrations of the body surface detected by the vibration sensor 2 are converted to an electrical signal and input to the stethoscope filter 4, which can output a signal corresponding to the frequency characteristics of the stethoscope 100.

Since a user can hear Korotkoff sounds by using the electronic stethoscope 10, it is possible to measure blood pressure based on a method in accordance with the Korotkoff method by referring to the cuff pressure value. Therefore, when measuring blood pressure, the electronic stethoscope 10 can be used in the same manner as a mechanical stethoscope.

A program for realizing all or some of the functions of the electronic blood pressure monitor 1 of the present invention may be recorded on a computer-readable storage medium, and the program recorded on this recording medium may be read into a computer system and executed, whereby the process is performed. Note that “computer system” here includes an operating system (OS) or hardware such as peripheral devices.

Furthermore, the “computer system” includes a WWW system provided with a homepage providing environment. The “computer readable recording medium” refers to a portable medium such as a flexible disk, a magneto-optical disc, a ROM, or a CD-ROM, and a storage device such as a hard disk housed in a computer system. Moreover, the “computer-readable recording medium” also includes a medium which holds a program for a specific time, such as volatile memory (RAM) inside a computer system that is a server or a client when the program is transmitted via a network such as the Internet or a communication line such as a telephone line.

The aforementioned program may be transmitted from a computer system having this program stored in a storage device thereof to another computer system via a transmission media or by transmission waves in the transmission media. The term “transmission media” that transmits the program includes a media having a function for transferring information, such as a network (communication network) such as the Internet, or a communication line (communication cable) such as a telephone line. In addition, the program may be for realizing a part of the aforementioned functions. Furthermore, the program may be a so-called differential file (differential program), whereby the functions described above can be realized by combination with programs that are already recorded in the computer system.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An electronic blood pressure monitor comprising:
a vibration sensor that comprises a film shape, the vibration sensor detecting vibrations of a body surface, the vibration sensor converting the detected vibrations to an electrical signal corresponding to pressure generated in a thickness direction of the vibration sensor to output the electrical signal; and
a stethoscope filter that passes a signal of a first predetermined frequency band among the output electrical signal, the first predetermined frequency band being determined based on a frequency characteristic of a stethoscope.

2. The electronic blood pressure monitor according to claim 1, wherein the vibration sensor detects the vibrations of the body surface by occurrence of pressure in the thickness direction of the vibration sensor.

3. The electronic blood pressure monitor according to claim 1, wherein the stethoscope filter comprises a first filter unit that passes a signal of a second predetermined frequency band, the second predetermined frequency band being determined based on a characteristic of a chest piece of the stethoscope.

4. The electronic blood pressure monitor according to claim 3,
wherein the chest piece of the stethoscope comprises a diaphragm and an internal air chamber formed between the diaphragm and a tube connected with the chest piece, and
the first filter unit passes the signal of the second predetermined frequency band based on at least one of an inertial element of the diaphragm, an elastic element of the diaphragm, a resistive element of the diaphragm, an elastic element of the internal air chamber, and a resistive element of the internal air chamber.

5. The electronic blood pressure monitor according to claim 4,
wherein the chest piece of the stethoscope further comprises a vent hole that brings the internal air chamber and outside into communication, and
the first filter unit passes the signal of the second predetermined frequency band based on a resistive element of the vent hole.

6. The electronic blood pressure monitor according to claim 1, wherein the stethoscope filter comprises a second filter unit that passes a signal of a third predetermined frequency band, the third predetermined frequency band being determined based on a characteristic of a tube connecting ear tips of the stethoscope and a chest piece of the stethoscope.

7. The electronic blood pressure monitor according to claim 6, wherein the second filter unit passes the signal of the third predetermined frequency band based on at least one of an elastic element of a medium and an inertial element of the medium, the medium transmitting sounds in a space formed by one end portion and the other end portion of a surface along an inner diameter of the tube.

8. The electronic blood pressure monitor according to claim 1, further comprising:
a loudness filter unit that passes a signal of a fourth predetermined frequency band among an electrical signal output by the stethoscope filter, the fourth predetermined frequency band being determined based on a human hearing characteristic.

9. The electronic blood pressure monitor according to claim 1,
wherein the stethoscope filter further comprises:
an input unit that acquires change information indicating a change of a circuit constant of a filter that passes a signal of a predetermined frequency band; and
a change unit that changes the circuit constant based on the acquired change information.

10. A blood pressure measuring method comprising:
detecting, by a vibration sensor that comprises a film shape, vibrations of a body surface;
converting, by the vibration sensor, the detected vibrations to an electrical signal corresponding to pressure generated in a thickness direction of the vibration sensor to output the electrical signal;
passing, by a stethoscope filter, a signal of a predetermined frequency band among the output electrical signal, the predetermined frequency band being determined based on a frequency characteristic of a stethoscope.

11. An electronic stethoscope comprising:
a vibration sensor that comprises a film shape, the vibration sensor detecting vibrations of a body surface, the vibration sensor converting the detected vibrations to an electrical signal corresponding to pressure generated in a thickness direction of the vibration sensor to output the electrical signal; and
a stethoscope filter that passes a signal of a predetermined frequency band among the output electrical signal, the predetermined frequency band being determined based on a frequency characteristic of a stethoscope.
